(19) United States
(12) Patent Application Publication
(10) Pub. No.: US 2026/0279106 A1
(43) Pub. Date: Sep. 17, 2026

(54) MEASUREMENT SYSTEM, MEASUREMENT DEVICE, AND MEASUREMENT METHOD

(71) Applicant: SONY SEMICONDUCTOR SOLUTIONS CORPORATION, KANAGAWA (JP)

(72) Inventors: BAHIEN CHU, KANAGAWA (JP); OSAMU KOZAKAI, KANAGAWA (JP); TORU TERASHIMA, KANAGAWA (JP)

(21) Appl. No.: 19/471,126

(22) PCT Filed: Mar. 13, 2024
(86) PCT No.: PCT/JP2024/009703

§ 371 Date: Sep. 30, 2025

(30) Foreign Application Priority Data
Apr. 10, 2023 (JP) 2023-063240

(51) Int. Cl. G06V 40/70; G01S 13/42; G01S 13/50; G01S 13/86; G06F 21/32; G06T 7/593; G06V 40/16

(52) U.S. Cl. CPC G06V 40/70; G01S 13/42; G01S 13/50; G01S 13/867; G06F 21/32; G06T 7/593; G06V 40/172; G06T 2207/10012; G06T 2207/10028; G06T 2207/30201; G06V 40/165

(57) ABSTRACT

To simultaneously measure biometric information while simultaneously perform personal authentication for a plurality of subjects. The present technology provides a measurement system that includes a sensor unit, a generation unit, an acquisition unit, and an association unit. The sensor unit transmits a first signal to a subject and receives a second signal reflected by the subject. The generation unit generates body motion information regarding body motion of the subject and first position information regarding a position of the subject on the basis of the first signal and the second signal. The acquisition unit acquires authentication information based on personal authentication of the subject, and second position information regarding the position of the subject. The association unit associates the body motion information and the authentication information with each other by collating the first position information and the second position information with each other.

### TECHNICAL FIELD

A technique according to the present disclosure (hereinafter also referred to as “the present technology”) relates to a measurement system, a measurement device, and a measurement method.

### BACKGROUND ART

In the field of measurement of human biometric information, measured biometric information and authentication information based on personal authentication of a human have been associated with each other.

For example, PTL 1 describes “a measurement subject identification method in which vital data and personal identification information are stored in a memory with the vital data and the personal identification information being correlated with each other”.

### CITATION LIST

### Patent Literature

PTL 1: Japanese Unexamined Patent Application Publication No. 2018-23768

### SUMMARY OF THE INVENTION

### Technical Problem

However, in the existing personal authentication described in, for example, PTL 1, it is difficult to simultaneously measure biometric information while simultaneously performing personal authentication for a plurality of subjects.

In view of the above, it is a main object of the present technology to provide a measurement system, a measurement device, and a measurement method in each of which biometric information is simultaneously measured while personal authentication is simultaneously performed for a plurality of subjects.

### Solution to Problem

The technology provides
a measurement system including:
a sensor unit that transmits a first signal to a subject and receives a second signal reflected by the subject;
a generation unit that generates body motion information regarding body motion of the subject and first position information regarding a position of the subject on the basis of the first signal and the second signal;
an acquisition unit that acquires authentication information based on personal authentication of the subject, and second position information regarding the position of the subject; and
an association unit that associates the body motion information and the authentication information with each other by collating the first position information and the second position information with each other.

The first position information may include at least information regarding a distance to the subject and information regarding a direction of the subject.

Each of the first signal and the second signal may be a signal that is of a millimeter-wave band and has a frequency varying over time.

The sensor unit may include a plurality of reception antennas each receiving the second signal.

The second position information may include at least information regarding a direction of the subject, and
the acquisition unit may acquire the information regarding the direction of the subject on the basis of an image obtained through imaging of the subject.

The acquisition unit may refrain from acquiring the second position information of the subject for whom the personal authentication has not been performed.

The second position information may include at least information regarding a distance to the subject and information regarding a direction of the subject, and
the acquisition unit may perform imaging of the subject to obtain a plurality of stereo camera images having parallax, and acquire the information regarding the distance to the subject and the information regarding the direction of the subject on the basis of the stereo camera images.

The second position information may include at least information regarding a distance to the subject and information regarding a direction of the subject, and
the acquisition unit may transmit a third signal to the subject, receive a fourth signal reflected by the subject, and acquire the information regarding the distance to the subject and the information regarding the direction of the subject on the basis of the third signal and the fourth signal.

The acquisition unit may include a single camera and a ToF sensor.

The acquisition unit may acquire the authentication information by performing face recognition of the subject.

The generation unit and the association unit may be coupled to each other via an information communication network.

The measurement system may further include a biometric information generation unit that generates biometric information of the subject on the basis of parameter information and the body motion information. The parameter information may be adapted to generate the first signal.

The generation unit and the biometric information generation unit may be coupled to each other via an information communication network.

The parameter information and the body motion information may be separately transmitted to the biometric information generation unit via an information communication network.

An analysis unit may be further provided that analyzes the biometric information generated by the biometric information generation unit.

The measurement system may further include
a display unit that displays an image acquired by the acquisition unit through imaging, and
the display unit may display the authentication information at a position at which the subject is displayed.

The acquisition unit may follow a movement of the subject.

The measurement system may further include an estimation unit that estimates biometric information on the basis of image data acquired by the acquisition unit through imaging.

In addition, the present technology provides
a measurement apparatus including:
a sensor unit that transmits a first signal to a subject and receives a second signal reflected by the subject;
a generation unit that generates body motion information regarding body motion of the subject and first position information regarding a position of the subject on the basis of the first signal and the second signal; and
an acquisition unit that acquires second position information regarding the position of the subject, and authentication information based on personal authentication of the subject.

In addition, the present technology provides
a measurement method including:
transmitting a first signal to a subject and receiving a second signal reflected by the subject;
generating body motion information regarding body motion of the subject and first position information regarding a position of the subject on the basis of the first signal and the second signal;
acquiring second position information regarding the position of the subject, and authentication information based on personal authentication of the subject; and
associating the body motion information and the authentication information with each other by collating the first position information and the second position information with each other.

According to the present technology, it is possible to simultaneously measure the biometric information while simultaneously performing the personal authentication for a plurality of subjects. It is to be noted that the effects described here are not necessarily limiting, and any of effects described in the present disclosure may be achieved.

### BRIEF DESCRIPTION OF THE DRAWINGS

FIG. 1 is a block diagram illustrating a configuration example of a measurement system 1000 according to an embodiment of the present technology.

FIG. 2 is a block diagram illustrating a configuration example of a measurement system 1000a according to an embodiment of the present technology.

FIG. 3A is a schematic diagram illustrating a configuration example of a millimeter-wave radar device. FIG. 3B is a graph illustrating variation in a signal to be processed by the millimeter-wave radar device.

FIG. 4 is a schematic diagram of a process flow of the millimeter-wave radar device.

FIG. 5 is a flowchart illustrating an example of a procedure of the measurement system 1000 according to the embodiment of the present technology.

FIG. 6 is an example of image data acquired by an acquisition unit 3 according to an embodiment of the present technology.

FIG. 7 is a block diagram illustrating a configuration example of a measurement system 1000b according to an embodiment of the present technology.

FIG. 8 is a flowchart illustrating an example of the procedure of the measurement system 1000 according to the embodiment of the present technology.

FIG. 9 is a block diagram illustrating a configuration example of a measurement system 1000c according to an embodiment of the present technology.

FIG. 10 is a block diagram illustrating a configuration example of a measurement system 1000d according to an embodiment of the present technology.

FIG. 11 is a block diagram illustrating a configuration example of a measurement system 1000e according to an embodiment of the present technology.

FIG. 12 is a block diagram illustrating a configuration example of a measurement system 1000f according to an embodiment of the present technology.

FIG. 13 is a block diagram illustrating a configuration example of a measurement system 1000g according to an embodiment of the present technology.

FIG. 14 is a block diagram illustrating a configuration example of a measurement system 1000h according to an embodiment of the present technology.

FIG. 15 is an example of an image to be displayed by a display unit 8 according to an embodiment of the present technology.

FIG. 16 is a flowchart illustrating an example of the procedure of the measurement system 1000 according to the embodiment of the present technology.

FIG. 17 is a block diagram illustrating a configuration example of a measurement system 1000i according to an embodiment of the present technology.

FIG. 18 is a block diagram illustrating a configuration example of a measurement device 100 according to an embodiment of the present technology.

FIG. 19 is a flowchart illustrating an example of a procedure of a measurement method according to an embodiment of the present technology.

### MODES FOR CARRYING OUT THE INVENTION

Hereinafter, preferred embodiments for carrying out the present technology will be described with reference to the drawings. It is to be noted that the embodiments described below are examples of representative embodiments of the present technology, and do not serve to limit the scope of the present technology. Further, the present technology may combine any of the following embodiments and modification examples thereof.

In the following description of the embodiments, the configuration is sometimes described with terms with “almost”, such as almost parallel or almost orthogonal. For example, almost parallel means not only being perfectly parallel, but also means being substantially parallel, i.e., including a state that is deviated from a perfect parallel state by, for example, several percent. This similarly applies to other terms with “almost”. In addition, the drawings are schematic diagrams, and are not necessarily illustrated exactly accurately. The scale of the drawings is exaggerated for clarity of the features of the technology. Therefore, it is to be noted that the scale of the drawings and the scale of the actual device are not necessarily the same as each other.

Unless otherwise specified, in the drawings, “upper” means an up direction or an upper side in the drawings, “lower” means a down direction or a lower side in the drawings, “left” means a left direction or a left side in the drawings, and “right” means a right direction or a right side in the drawings. Further, in the drawings, the same or equivalent components or members are denoted with the same reference numerals, and redundant description thereof will be omitted.

The description is given in the following order.
1. First Embodiment of Present Technology (Example 1 of Measurement System)
   (1) Overview
   (2) Millimeter-Wave Radar Device
   (3) Process Flow
2. Second Embodiment of Present Technology (Example 2 of Measurement System)
3. Third Embodiment of Present Technology (Example 3 of Measurement System)
4. Fourth Embodiment of Present Technology (Example 4 of Measurement System)
5. Fifth Embodiment of Present Technology (Example 5 of Measurement System)
6. Sixth Embodiment of Present Technology (Example 6 of Measurement System)
7. Seventh Embodiment of Present Technology (Example 7 of Measurement System)
8. Eighth Embodiment of Present Technology (Example 8 of Measurement System)
9. Ninth Embodiment of Present Technology (Example 9 of Measurement System)
10. Tenth Embodiment of Present Technology (Example 10 of Measurement System)
11. Eleventh Embodiment of Present Technology (Example 11 of Measurement System)
12. Twelfth Embodiment of Present Technology (Example of Measurement Device)
13. Thirteenth Embodiment of Present Technology (Example of Measurement Method)

### 1. First Embodiment of Present Technology (Example 1 of Measurement System)

### [(1) Overview]

In the technical field of measurement of biometric information such as a body temperature, a heart rate, or a pulse rate of a subject, personal authentication of the subject has been performed. To perform such personal authentication, active authentication may be performed in which, for example, the subject inputs an ID and a password into a device. Alternatively, passive authentication may be performed by, for example, recognition of the face, the fingerprint, or the like of the subject. Associating authentication information based on the personal authentication and the measured biometric information with each other makes it possible to manage biometric information of a specific individual.

In a case of measuring biometric information for subjects one by one, the measurement may be performed by an existing method. However, it is difficult to simultaneously measure the biometric information while simultaneously performing personal authentication for a plurality of subjects by the existing method. For example, it is possible to achieve the above with use of a plurality of authentication devices and a plurality of measurement devices; however, this results in high cost.

Alternatively, it is possible to measure the biometric information of a plurality of subjects with use of a single authentication device and a single measurement device by determining the measurement order of the subjects. However, this requires identifying the subject on the basis of measurement timing, which leaves an issue in user-friendliness. In addition, one-by-one measurement increases measurement time.

To address the above, the present technology provides a measurement system that simultaneously measures biometric information while simultaneously performing personal authentication for a plurality of subjects. Specifically, provided is a measurement system that includes a sensor unit, a generation unit, an acquisition unit, and an association unit. The sensor unit transmits a first signal to a subject and receives a second signal reflected by the subject. The generation unit generates body motion information regarding body motion of the subject and first position information regarding a position of the subject on the basis of the first signal and the second signal. The acquisition unit acquires authentication information based on personal authentication of the subject, and second position information regarding the position of the subject. The association unit associates the body motion information and the authentication information with each other by collating the first position information and the second position information with each other.

A configuration example of the measurement system will be described with reference to FIG. 1. FIG. 1 is a block diagram illustrating a configuration example of a measurement system 1000 according to an embodiment of the present technology. As illustrated in FIG. 1, the measurement system 1000 includes a sensor unit 1, a generation unit 2, an acquisition unit 3, and an association unit 4.

The sensor unit 1 transmits a first signal to a subject, and receives a second signal reflected by the subject. The first signal and the second signal are not particularly limited in kind of signal as long as the first signal and the second signal allow for acquisition at least of a distance to the subject or a direction in which the subject is present.

The generation unit 2 generates, on the basis of the first signal and the second signal, body motion information regarding body motion of the subject and first position information regarding a position of the subject. The body motion information includes information regarding minute motion in a body tissue of the subject, minute motion of a body surface of the subject, or the like. The first position information includes at least information regarding the distance to the subject and information regarding the direction of the subject.

The acquisition unit 3 acquires authentication information based on personal authentication, and second position information regarding the position of the subject. The second information acquired by a method different from that used by the sensor unit 1 includes at least the information regarding the direction of the subject.

Each of the first position information and the second position information includes at least the information regarding the direction in which the subject is present. This allows the association unit 4 to associate the body motion information and the authentication information with each other by collating the first position information and the second position information with each other. For example, it is possible to associate the body motion information generated by the generation unit 2 and the authentication information acquired by the acquisition unit 3 with each other by collating respective directions of a plurality of subjects generated by the generation unit 2 and the respective directions of the plurality of subjects acquired by the acquisition unit 3 with each other.

Each of the first position information and the second position information may further include the information regarding the distance to the subject. Collating on the basis of the direction of the subject and the distance to the subject makes it possible to associate the body motion information and the authentication information with each other with higher accuracy.

According to the present technology, it is possible to simultaneously measure the biometric information while simultaneously performing the personal authentication for a plurality of subjects. This improves user-friendliness and improves measurement efficiency. This effect is achieved similarly also by other embodiments to be described below. Therefore, in the description of the other embodiments, the effect may not be described again.

For example, it is possible to use the present technology by installing the sensor unit 1 and the acquisition unit 3 in sporting clubs, nursing homes, hospitals, personal homes, etc.

It is to be noted that all of the components of the measurement system 1000 may be included in a single device, or a part of the components of the measurement system 1000 may be included in a cloud server. For example, the sensor unit 1, the generation unit 2, and the acquisition unit 3 may be included in a single device, and the association unit 4 may be included in the cloud server.

The association unit 4 is implementable by, for example, a computing unit such as a CPU reading a program. The program may be stored in a device including the sensor unit 1 and the acquisition unit 3, or may be stored in a cloud server.

The program may be stored and provided to a computer, using any of various types of non-transitory computer readable media (non-transitory computer readable media). The non-transitory computer readable media include various types of tangible storage media (tangible storage media). Examples of the non-transitory computer readable media include a magnetic recording medium (such as a flexible disk, a magnetic tape, or a hard disk drive), a magneto-optical recording medium (such as a magneto-optical disk), a compact disc read only memory (CD-ROM), a CD-R, a CD-R/W, a semiconductor memory (such as a mask ROM, a programmable ROM (PROM), an erasable PROM (EPROM), a flash ROM, and a random access memory (RAM).

Alternatively, the above-described program may be supplied to a computer by any of various types of transitory computer readable media (transitory computer readable media). Examples of the transitory computer readable medium include an electrical signal, an optical signal, and an electromagnetic wave. The transitory computer readable medium is configured to allow the above-described program to be supplied to a computer via a wired communication path such as an electric wire or an optical fiber, or via a wireless communication path.

### [(2) Millimeter-Wave Radar Device]

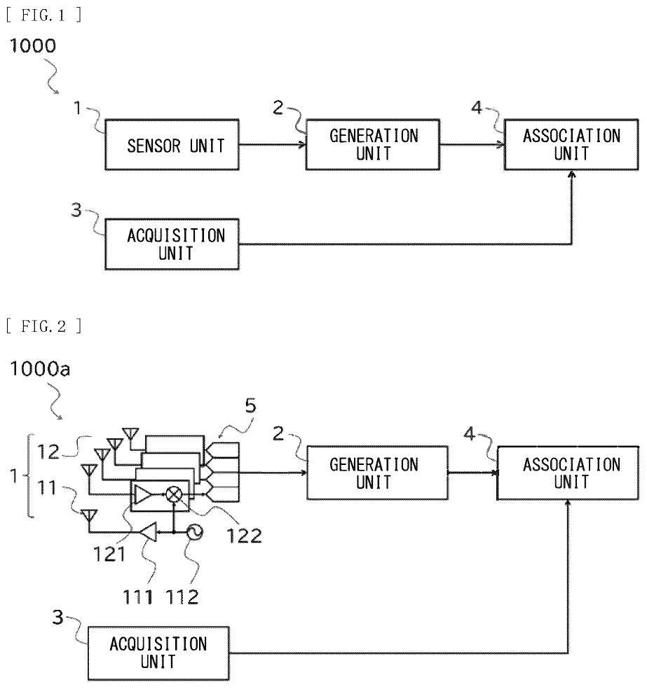

Each of the first signal and the second signal may be a signal that is of a millimeter-wave band and has a frequency varying over time. The signal of the millimeter-wave band may be, for example, an electromagnetic wave, an ultrasonic wave, or the like. A configuration example of a measurement system 1000a in a case where the signal of the millimeter-wave band is an electromagnetic wave will be described with reference to FIG. 2. FIG. 2 is a block diagram illustrating a configuration example of the measurement system 1000a according to an embodiment of the present technology. As illustrated in FIG. 2, the measurement system 1000a includes the sensor unit 1, the generation unit 2, the acquisition unit 3, and the association unit 4.

The sensor unit 1 and the generation unit 2 may be components of, for example, a millimeter-wave radar device. The sensor unit 1 includes a transmission antenna 11 and a reception antenna 12. The transmission antenna 11 transmits the first signal to the subject. The reception antenna 12 receives the second signal reflected by the subject. It is to be noted that the transmission antenna 11 and the reception antenna 12 are preferably included in the same device; however, the transmission antenna 11 and the reception antenna 12 may be included in respective separate devices.

The transmission antenna 11 is coupled to a PA (Power Amplifier) 111. The PA 111 is a kind of electronic circuit used to amplify a signal. When a small input signal generated by a signal source 112 is inputted to the PA 111, the PA 111 amplifies it to a large output signal, and the output signal is transmitted to the transmission antenna 11. The PA 111 includes various elements including, without limitation, a transistor, a vacuum tube, a semiconductor element, and the like to amplify the signal.

The transmission antenna 11 transmits the first signal, which is a chirp signal, to the subject. When the first signal applied on a surface of a living body of the subject is reflected, the first signal changes to the second signal due to characteristics of scattering, absorption, or the like of the surface of the living body. The reception antenna 12 receives the second signal, which is a chirp signal reflected by the subject.

The sensor unit 1 includes, for example, an LNA (Low Noise Amplifier) 121 and a mixer 122. The LNA 121 is an amplifier configured to amplify a part of an input signal other than the noise of the input signal. Because the millimeter-wave radar receives the second signal that has been reflected, the millimeter-wave radar needs to receive a very weak signal. The LNA 121 is used to amplify such a weak signal. The mixer 122 combines the first signal and the second signal to generate an IF (Intermediate Frequency) signal. The IF signal is subjected to AD conversion by an AD converter 5 to be a digital signal.

The generation unit 2 analyzes the IF signal to generate the body motion information regarding the body motion of the subject and the first position information regarding the position of the subject.

The acquisition unit 3 acquires the authentication information based on the personal authentication of the subject and the second position information regarding the position of the subject. The association unit 4 is configured to associate the body motion information and the authentication information with each other by collating the first position information and the second position information with each other.

A process of the millimeter-wave radar device constituting the sensor unit 1 and the generation unit 2 will be described with reference to FIG. 3. FIG. 3A is a schematic diagram illustrating a configuration example of the millimeter-wave radar device.

As illustrated in FIG. 3A, the transmission antenna 11 transmits a first signal W1 to the subject. The first signal W1 is a chirp signal. The reception antenna 12 receives a second signal W2. The second signal W2 is a chirp signal reflected by the subject. The chirp signal is a signal that is of a millimeter-wave band and has a frequency varying over time. That is, each of the first signal and the second signal is a signal that is of a millimeter-wave band and has a frequency varying over time.

The mixer 22 generates an IF signal W3 by combining the first signal and the second signal. The IF signal is subjected to AD conversion and thus converted into a digital signal by the AD converter 5, and is thereafter transformed into a signal of a frequency domain by fast Fourier transform (Fast Fourier Transform: FFT).

An example of variation in the first signal and the second signal will be described with reference to FIG. 3B. FIG. 3B is a graph illustrating an example of variation in signals which the millimeter-wave radar device is to process. In a left part of FIG. 3B, a horizontal axis represents time, and a vertical axis represents a frequency of a signal.

First, the first signal W1 transmitted to the subject varies over time from an initial frequency f₀ toward a target frequency f₁. The time it takes for the first signal W1 to vary from the frequency f₀ to the frequency f₁ is a chirp time Tc. A difference between the frequency f₀ and the frequency f₁ is a sweep bandwidth BW.

Thereafter, the second signal W2 reflected by the subject also varies in frequency over time, in a manner similar to that of the first signal W1. Time from a timing when the signal is transmitted from the millimeter-wave radar device to a timing when the signal is reflected by the subject and returns to the millimeter-wave radar device is a delay time T.

A frequency fir of the IF signal W3 in which the first signal W1 and the second signal W2 are mixed, and the distance to the subject correlate. Specifically, a distance d from the measurement system 1000 to the subject is calculatable on the basis of the following Expression (1). It is to be noted that “c” represents a speed of a radio wave.

$$\begin{matrix}[ Math . 1 ] & \\ d = \frac{{T}_{C} \times {f}_{IF}}{2 \times c \times BW} & ( 1 )\end{matrix}$$

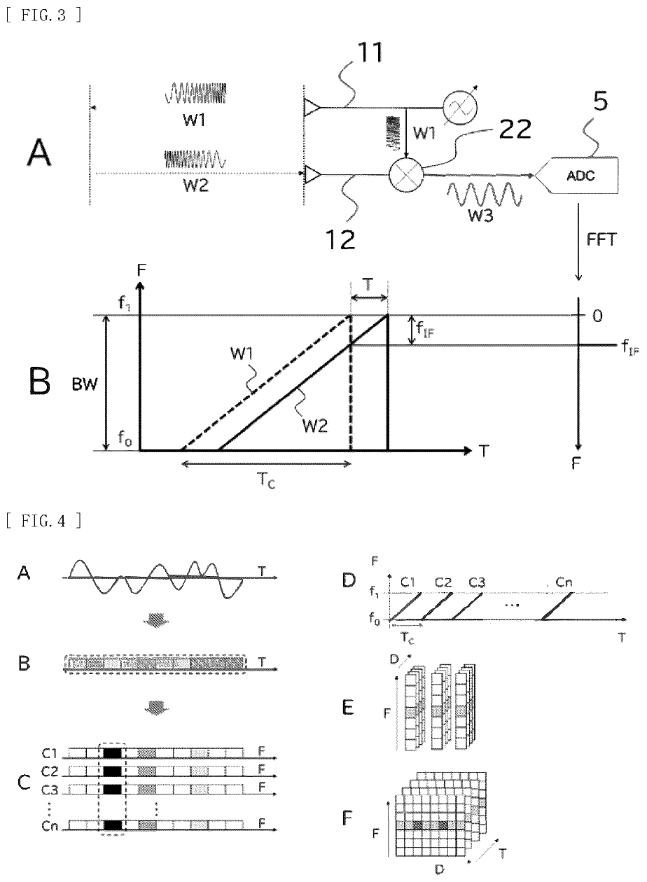

Next, a flow of the process of the millimeter-wave radar device will be described with reference to FIG. 4. FIG. 4 is a schematic diagram regarding the flow of the process of the millimeter-wave radar device.

FIG. 4A illustrates an example of the IF signal, which is an analog signal. In FIG. 4A, a horizontal axis represents time. The IF signal, which is the digital signal illustrated in FIG. 4B, is obtained by performing AD conversion on the analog signal. Frequency components are obtained as illustrated in FIG. 4C with a horizontal axis as a frequency, by analyzing the IF signal, using, for example, range FFT (Fast Fourier Transform) as a frequency analysis algorithm.

Meanwhile, FIG. 4D illustrates variation over time in the first signal transmitted to the subject. In the graph illustrated in FIG. 4D, a horizontal axis represents time, and a vertical axis represents a frequency. As illustrated in FIG. 4D, the first signal transmitted to the subject varies over time from the initial frequency f₀ to the target frequency f₁, and thereafter returns to the initial frequency f₀. Further, the first signal varies over time again from the initial frequency f₀ to the target frequency f₁. The frequency of the first signal thus repeatedly varies. Therefore, as illustrated in FIG. 4C, frequency components corresponding to a unit change of the first signal are obtained.

In the frequency components illustrated in FIG. 4C, a darker color indicates a higher observation level. The second signal is reflected by various objects in the space and the second signals thus reflected are received. However, a frequency component that is high in observation level can be estimated to be a frequency component based on the signal reflected by the subject. Because the distance to the subject and the frequency of the IF signal correlate as described above, it is possible to estimate the distance to the subject by extracting a frequency component that is high in observation level.

Further, in order to associate the body motion information and the authentication information with each other with higher accuracy, it is preferable that the direction in which the subject is present can be estimated in addition to the distance to the subject. In order to achieve this, the sensor unit 1 preferably includes a plurality of reception antennas that receives the second signal. This makes it possible to estimate the direction in which the subject is present, on the basis of a phase difference between a plurality of second signals received within the same chirp time. In FIG. 4E, a vertical axis represents a frequency. The frequency components based on the signals received by the reception antennas are arranged from a front side toward a back side. In each of unit variations C1 to C3, four kinds of frequency components based on the second signal received by the four reception antennas are illustrated. It is possible to estimate the direction in which the subject is present, on the basis of the four kinds of frequency components. It is to be noted that the number of the reception antennas is not limited to four, and may be any number that is two or more.

The diagram illustrated in FIG. 4E can be converted as illustrated in FIG. 4F. In FIG. 4F, a vertical axis represents a frequency and a horizontal axis represents a direction. Frequency components corresponding to the chirp time are arranged from the front side toward the back side. That is, the frequency components vary over time from the front side toward the back side. It is thus possible to estimate the distance to the subject and the direction of the subject, and to observe variation over time. Accordingly, it is possible to observe the body motion of the subject, such as bulging of the lung of the subject.

It is to be noted that a method of analyzing the IF signal is not limited to the above. For example, the distance to the subject may be measured by a pulse compression technique. The pulse compression technique is a technique of measuring a distance with high resolution by using a pulse signal having a short pulse width. A short pulse width is obtainable by correlating a transmitted pulse signal and a received reflection signal. It is thus possible to measure the distance with high accuracy.

Alternatively, the distance to the subject may be measured by a biometric information analysis technique. The biometric information analysis technique is a technique of observing a structure, a function, or the like of a body by analyzing a weak electromagnetic wave reflected by a body tissue. It is possible to observe a structure, a function, or the like of the tissue by transmitting an electromagnetic wave to the body tissue and analyzing a reflection signal.

### [(3) Process Flow]

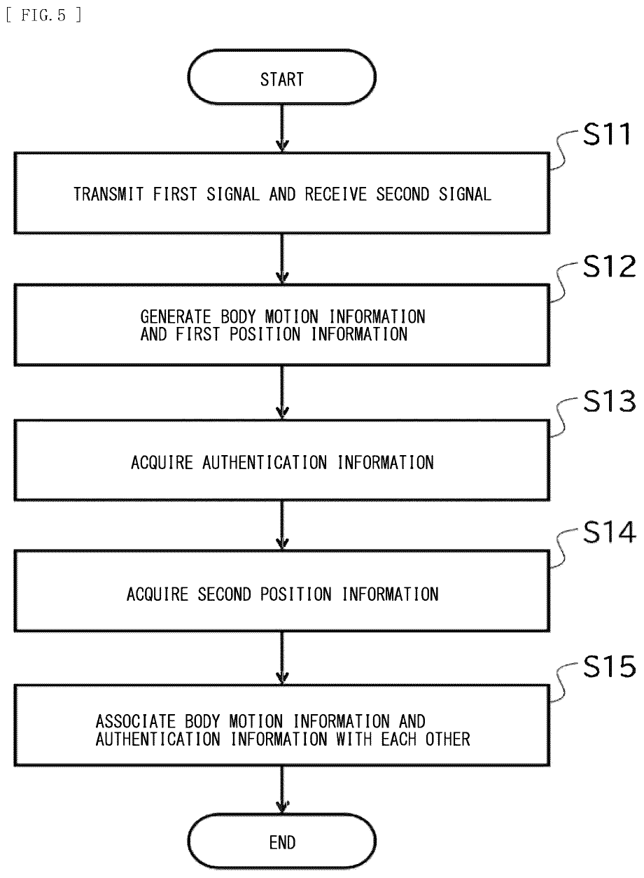

An example of a procedure of the measurement system 1000 according to the embodiment of the present technology will be described with reference to FIG. 5. FIG. 5 is a flowchart illustrating an example of the procedure of the measurement system 1000 according to the embodiment of the present technology.

First, in step S11, the sensor unit 1 transmits the first signal to the subject, and receives the second signal reflected by the subject.

Thereafter, in step S12, the generation unit 2 generates the body motion information regarding the body motion of the subject and the first position information regarding the position of the subject on the basis of the first signal and the second signal. In a case where a plurality of subjects is present, the generation unit 2 generates the body motion information regarding the body motion of each of the subjects and the first position information regarding the position of each of the subjects.

The body motion information includes, for example, information regarding minute body motion of the subject, such as bulging of the lung of the subject. The first position information includes at least the information regarding the distance to the subject and the information regarding the direction of the subject.

Thereafter, in step S13, the acquisition unit 3 acquires the authentication information based on the personal authentication of the subject. In a case where a plurality of subjects is present, the acquisition unit 3 acquires the authentication information based on the personal authentication of each of the subjects.

The authentication information may be, for example, the name of the subject. When the acquisition unit 3 is, for example, a camera, the acquisition unit 3 is configured to acquire the authentication information through face recognition of the subject. The acquisition unit 3 photographs the face of the subject, analyzes features of the face, and thus performs the personal authentication of the subject. For example, the acquisition unit 3 may detect features including, without limitation, the face outline, the eye, the mouth, the nose, and the like from an image of the face and analyze positional relationships, shapes, and the like of the feature points to perform the personal authentication.

Information for identifying an individual may be stored, for example, as a database. Such a database holds, for example, an image of the face and feature point data of the face of a registered person. At the time of authentication, identification, or the like the personal authentication of the person is performed by analyzing the image of the face captured by the camera and collating it with the information in the database.

The acquisition unit 3 preferably refrains from acquiring the second position information of the subject for whom the personal authentication has not been performed. Accordingly, the body motion information and the authentication information are not associated with each other, in step S15 to be described later, for the subject for whom the personal authentication has not been performed. As a result, it is possible to prevent erroneous association. Further, it is not necessary to generate, in step S12, information such as the body motion information regarding the body motion of the subject, for the subject for whom the personal authentication has not been performed. Collecting bare minimum information makes it possible to appropriately protect personal information, privacy, and the like. Further, not generating unnecessary information makes it possible to improve measurement efficiency.

Thereafter, in step S14, the acquisition unit 3 acquires the second position information regarding the position of the subject. In a case where a plurality of subjects is present, the second position information regarding the position of each of the subjects is acquired.

The second position information includes at least the information regarding the direction of the subject. That is, the acquisition unit 3 is able to acquire the information regarding the direction of the subject on the basis of the image obtained through imaging of the subject. The acquisition unit 3 may be, for example, a camera or the like. It is possible for the acquisition unit 3 to acquire the information regarding the direction of the subject by focusing on the characteristics of the subject analyzed in step S13.

This will be described further with reference to FIG. 6. FIG. 6 illustrates an example of image data acquired by the acquisition unit 3 according to the embodiment of the present technology. For example, two subjects P1 and P2 are captured in the image, as illustrated in FIG. 6.

In this diagram, the faces of the subjects P1 and P2 are detected and in focus. It is possible to perform the personal authentication of each of the subject P1 and the subject P2 and to detect the direction in which each of the subject P1 and the subject P2 is present.

A method of detecting the direction in which the subject is present on the basis of the image data is not particularly limited. For example, the direction in which the subject is present is detectable by detecting feature points of the face or the like of the subject, and analyzing a positional relationship between the feature points. For example, Haar Cascade, HOG (Histogram of Oriented Gradients), deep learning, or the like may be used to detect the feature points of the face.

Alternatively, in a case where the subject is moving, the direction in which the subject is present is detectable by detecting a movement direction. The movement direction of the subject is detectable by analyzing a movement amount of a pixel in the image.

Alternatively, the direction of the subject is detectable using color information of the subject. For example, in shooting using a green back screen, because the background is green, the direction of the subject is detectable unless the subject is green.

It is to be noted that the above-described methods may be used in combination.

The description will be given with reference to FIG. 5 again. Thereafter, in step S15, the association unit 4 associates the body motion information and the authentication information with each other by collating the first position information and the second position information with each other. Each of the first position information and the second position information includes at least the information regarding the direction in which the subject is present. It is therefore possible for the association unit 4 to associate the body motion information and the authentication information with each other on the basis of the information regarding the direction in which the subject is present.

It is possible to generate biometric information such as a heartbeat of the subject on the basis of the body motion information, and parameter information adapted to generate the first signal. The parameter information includes, for example, the chirp time Tc, the sweep bandwidth BW, or the like illustrated in FIG. 3.

According to the present technology, it is possible to simultaneously measure the biometric information while simultaneously performing the personal authentication for a plurality of subjects.

The contents described above regarding the measurement system according to the first embodiment of the present technology are applicable to other embodiments of the present technology unless there is any technical inconsistency.

### 2. Second Embodiment of Present Technology (Example 2 of Measurement System)

The second position information acquired by the acquisition unit 3 may include the information regarding the distance to the subject in addition to the information regarding the direction in which the subject is present. A method of acquiring the information regarding the distance to the subject is not particularly limited. For example, a stereo camera image may be used.

That is, the second position information may include at least the information regarding the distance to the subject and the information regarding the direction of the subject. The acquisition unit 3 may perform imaging of the subject to obtain a plurality of stereo camera images having parallax, and may acquire the information regarding the distance to the subject and the information regarding the direction of the subject on the basis of the stereo camera images.

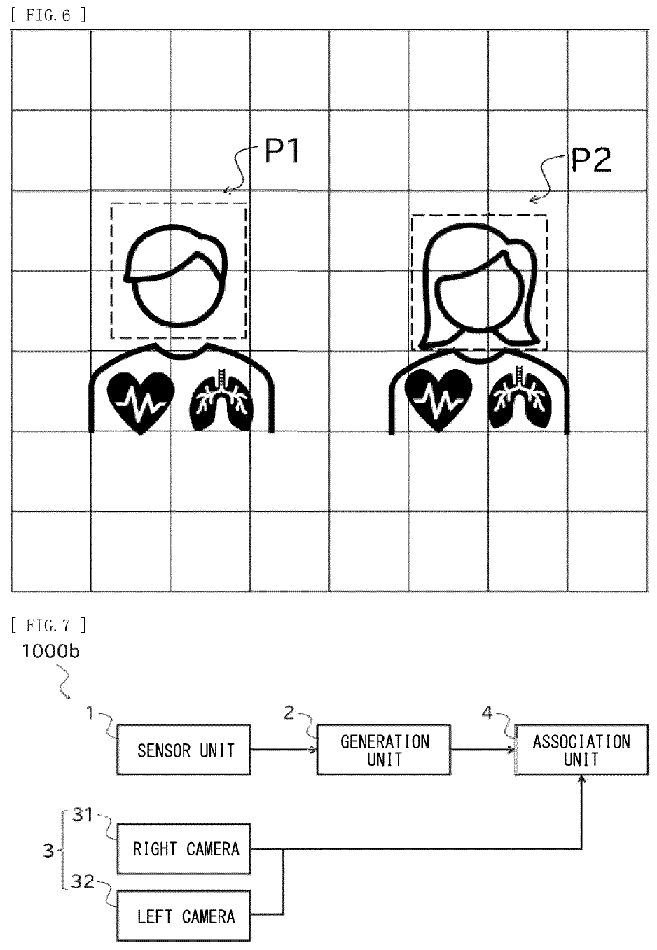

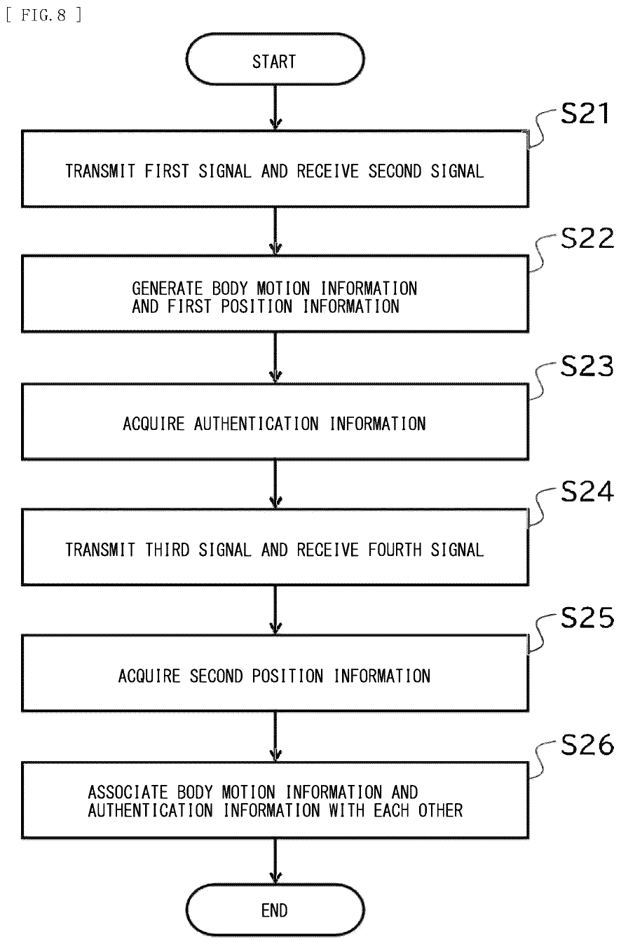

This will be described with reference to FIG. 7. FIG. 7 is a block diagram illustrating a configuration example of a measurement system 1000b according to an embodiment of the present disclosure. As illustrated in FIG. 7, the acquisition unit 3 is a stereo camera including a right camera 31 and a left camera 32. The left camera 32 acquires an image from the left side of the subject, and the right camera 31 acquires an image from the right side of the subject. It is thus possible to generate a subtle difference in angle to acquire the information regarding the distance to the subject.

The association unit 4 is configured to associate the body motion information and the authentication information with each other on the basis of the information regarding the direction in which the subject is present and the information regarding the distance to the subject. It is thus possible for the association unit 4 according to the present embodiment to perform association with high accuracy as compared with the association unit 4 according to the embodiment that performs association on the basis of the information regarding the direction in which the subject is present.

The contents described above regarding the measurement system according to the second embodiment of the present technology are applicable to other embodiments of the present technology unless there is any technical inconsistency.

### 3. Third Embodiment of Present Technology (Example 3 of Measurement System)

A signal may be used as an example of the method of acquiring the information regarding the distance to the subject. That is, the second position information includes at least the information regarding the distance to the subject and the information regarding the direction of the subject. The acquisition unit 3 transmits a third signal to the subject, receives a fourth signal reflected by the subject, and acquires the information regarding the distance to the subject and the information regarding the direction of the subject on the basis of the third signal and the fourth signal.

An example of the procedure of the measurement system 1000 according to the present embodiment will be described with reference to FIG. 8. FIG. 8 is a flowchart illustrating the example of the procedure of the measurement system 1000 according to the embodiment of the present technology.

First, in step S21, the sensor unit 1 transmits the first signal to the subject, and receives the second signal reflected by the subject.

Thereafter, in step S22, the generation unit 2 generates the body motion information regarding the body motion of the subject and the first position information regarding the position of the subject on the basis of the first signal and the second signal. The first position information includes at least the information regarding the distance to the subject and the information regarding the direction of the subject.

Thereafter, in step S23, the acquisition unit 3 acquires the authentication information based on the personal authentication of the subject.

Thereafter, in step S24, the acquisition unit 3 transmits the third signal to the subject, and receives the fourth signal reflected by the subject.

Thereafter, in step S25, the acquisition unit 3 acquires the second position information on the basis of the third signal and the fourth signal. The second position information includes the information regarding the distance to the subject and the information regarding the direction of the subject.

A method of acquiring the information regarding the distance to the subject and the information regarding the direction of the subject is not particularly limited. For example, the acquisition unit may include an optical sensor. For example, the acquisition unit 3 may include a LiDAR (Light Detection and Ranging) as an example of the optical sensor. The LiDAR is a kind of sensor that measures a distance to an object by using laser light. The LiDAR is configured to emit the laser light (the third signal) and receive the light (the fourth signal) reflected by the object to measure the distance to the object, the shape of the object, or the like. The LiDAR is configured to acquire the measurement result as a point cloud of a three-dimensional space. It is thus possible for the acquisition unit 3 to detect the distance to the subject and the direction of the subject. In addition, because the LiDAR is configured to measure the shape of the object, for example, it is possible to perform the personal authentication by storing, for example, the shape of the face.

The LiDAR is configured to, for example, detect the distance to the subject and the direction of the subject by using triangulation, a flight of time, a phase shift, or the like. The LiDAR is configured to perform highly accurate measurement based on transmission and reception of the laser light, and is characterized in high accuracy as compared with other sensing techniques. In addition, it is possible to choose the wavelength of the laser light between ultraviolet rays, infrared rays, and the like. It is thus possible to perform measurement with higher accuracy by choosing an appropriate wavelength depending on a characteristic of a detection target.

The contents described above regarding the measurement system according to the third embodiment of the present technology are applicable to other embodiments of the present technology unless there is any technical inconsistency.

### 4. Fourth Embodiment of Present Technology (Example 4 of Measurement System)

As an example of the method of acquiring the information regarding the distance to the subject and the information regarding the direction of the subject, the acquisition unit 3 may include a single camera and a ToF (Time of Flight) sensor.

This will be described with reference to FIG. 9. FIG. 9 is a block diagram illustrating a configuration example of a measurement system 1000c according to an embodiment of the present technology. As illustrated in FIG. 9, the acquisition unit 3 includes a single camera 33 and a ToF sensor 34.

As described above regarding the other embodiments, the single camera 33 acquires the information regarding the direction in which the subject is present.

The ToF sensor 34 is a sensor that measures a distance by using a time of flight of light. The ToF sensor 34 measures the time of flight of light and measures the distance by emitting light from a light source such as an infrared ray LED and detecting, using the sensor, the light that has hit the subject. It is thus possible for the ToF sensor 34 to acquire the information regarding the distance to the subject.

As compared with other distance measurement techniques such as a laser range finder or a stereo camera, the ToF sensor 34 is less influenced by the color, the material, or the like of the measurement target, and is thus able to perform measurement at high speed and with high accuracy.

The contents described above regarding the measurement system according to the fourth embodiment of the present technology are applicable to other embodiments of the present technology unless there is any technical inconsistency.

### 5. Fifth Embodiment of Present Technology (Example 5 of Measurement System)

The biometric information is very important as personal information. Therefore, in a case where the biometric information is stored in a device, there is a risk that the data leaks out when the device itself is stolen or unauthorized access is made, for example, by hacking.

The biometric information is information reflecting a health condition and physical characteristics of the individual, and the leakage thereof can seriously affect privacy, security, and the like of the individual. For example, when heart rate data leaks out, the health condition, a lifestyle habit, or the like of the individual becomes known to the outside, which can affect employment, insurance application, and the like. In addition, because the heart rate data is also used for biometric authentication, in a case where the heart rate data leaks out, there is a possibility that the heart rate data is abused for unauthorized access or spoofing.

Therefore, it is necessary to pay sufficient attention in managing the body motion information that is convertible to the biometric information. Accordingly, association information in which the authentication information is associated with the body motion information convertible to the biometric information is preferably stored in a cloud system. This reduces the risk of the association information being stolen.

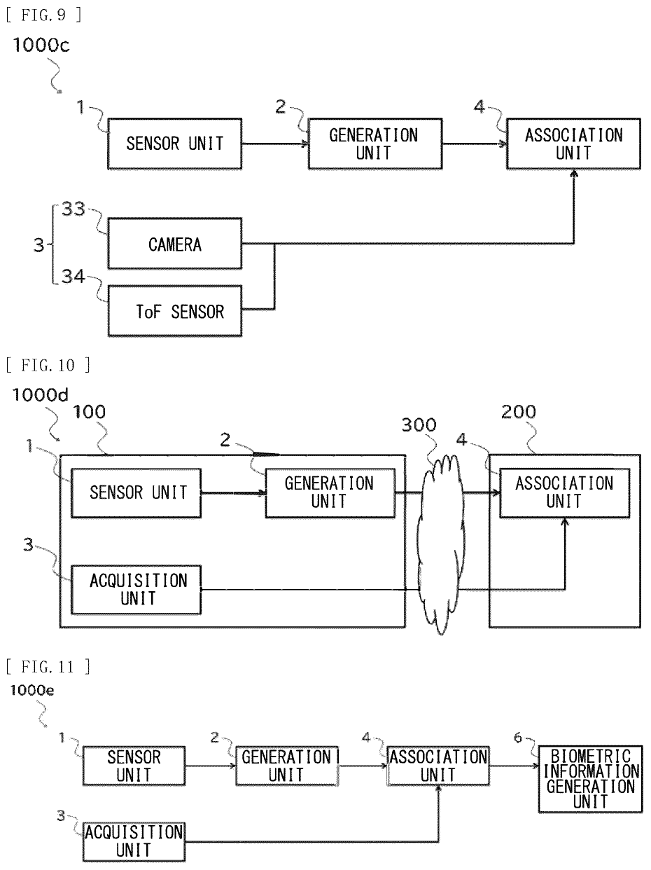

That is, it is preferable that the sensor unit 1, the generation unit 2, and the acquisition unit 3 be included in a single measurement device, and the association unit 4 be included in a cloud server. This will be described with reference to FIG. 10. FIG. 10 is a block diagram illustrating a configuration example of a measurement system 1000d according to an embodiment of the present technology.

As illustrated in FIG. 10, the sensor unit 1, the generation unit 2, and the acquisition unit 3 are included in a single measurement device 100. It is to be noted that the measurement device 100 is not necessarily one in number, and a plurality of measurement devices 100 may be provided. The sensor unit 1, the generation unit 2, and the acquisition unit 3 may be included in respective different measurement devices.

The association unit 4 is included in a cloud server 200. The generation unit 2 and the association unit 4 are coupled to each other via an information communication network 300. The body motion information and the authentication information are each transmitted to the cloud server 200 via the information communication network 300, and are thereafter associated with each other by the association unit 4 included in the cloud server 200. This prevents the association information from being stored in the measurement device 100, which reduces the risk of the association information in which the body motion information and the authentication information are associated with each other being stolen. The information communication network 300 includes, for example, a LAN (Local Area Network), a WAN (Wide Area Network), a VPN (Virtual Private Network), the Internet, a PAN (Personal Area Network), a SAN (Storage Area Network), or the like.

In this case, it is preferable that the body motion information and the authentication information be separately transmitted to the association unit 4 via the information communication network 300. This prevents the body motion information and the authentication information from being associated with each other by a third party even if either the body motion information or the authentication information is stolen by the third party through hacking during transmission. As a result, it is possible to reduce the risk of the personal information leaking out.

The contents described above regarding the measurement system according to the fifth embodiment of the present technology are applicable to other embodiments of the present technology unless there is any technical inconsistency.

### 6. Sixth Embodiment of Present Technology (Example 6 of Measurement System)

The measurement system 1000 according to the embodiment of the present technology is configured to generate the biometric information such as information regarding the heartbeat on the basis of the body motion information generated by the generation unit 2. This will be described with reference to FIG. 11. FIG. 11 is a block diagram illustrating a configuration example of a measurement system 1000e according to an embodiment of the present technology.

As illustrated in FIG. 11, the measurement system 1000e further includes a biometric information generation unit 6. The biometric information generation unit 6 generates the biometric information of the subject on the basis of the parameter information adapted to generate the first signal, and the body motion information. The parameter information is generated by the sensor unit 1. The parameter information includes, for example, the chirp time Tc, the sweep bandwidth BMW, or the like illustrated in FIG. 3.

According to the present technology, it is possible to simultaneously measure the biometric information while simultaneously performing the personal authentication for a plurality of subjects.

The contents described above regarding the measurement system according to the sixth embodiment of the present technology are applicable to other embodiments of the present technology unless there is any technical inconsistency.

### 7. Seventh Embodiment of Present Technology (Example 7 of Measurement System)

As described above, the biometric information is very important as personal information. Therefore, it is preferable that the sensor unit 1, the generation unit 2, and the acquisition unit 3 be included in a single measurement device, and the biometric information generation unit 6 be included in a cloud server.

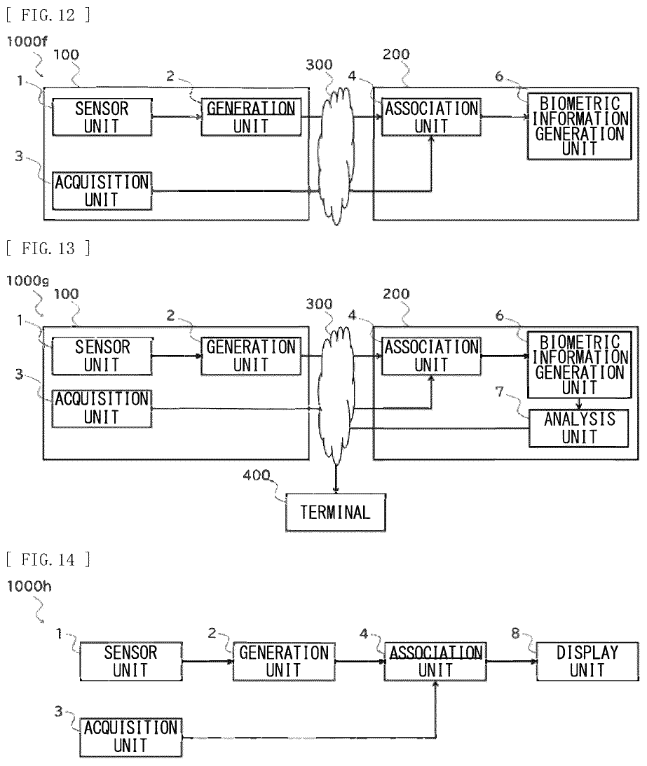

This will be described with reference to FIG. 12. FIG. 12 is a block diagram illustrating a configuration example of a measurement system 1000f according to an embodiment of the present technology.

As illustrated in FIG. 12, the sensor unit 1, the generation unit 2, and the acquisition unit 3 are included in a single measurement device 100. It is to be noted that the measurement device 100 is not necessarily one in number, and a plurality of measurement devices 100 may be provided. The sensor unit 1, the generation unit 2, and the acquisition unit 3 may be included in respective different measurement devices.

The association unit 4 and the biometric information generation unit 6 are included in the cloud server 200. The generation unit 2 and the biometric information generation unit 6 are coupled to each other via the information communication network 300.

This prevents the biometric information from being stored in the measurement device 100, which reduces the risk of the biometric information being stolen. In addition, this configuration makes it possible to update, on an as-needed basis, an algorithm of the biometric information generation unit 6 included in the cloud server 200. Accordingly, it is possible to generate the biometric information with the use of the latest algorithm for the biometric information acquired in the past, for example. As a result, it is possible to generate the biometric information with higher accuracy.

In this case, it is preferable that the parameter information and the body motion information be separately transmitted to the biometric information generation unit via the information communication network 300. This prevents the biometric information from being generated by a third party even if either the parameter information or the body motion information is stolen by the third party through hacking during transmission. As a result, it is possible to reduce the risk of the personal information leaking out.

The contents described above regarding the measurement system according to the seventh embodiment of the present technology are applicable to other embodiments of the present technology unless there is any technical inconsistency.

### 8. Eighth Embodiment of Present Technology (Example 8 of Measurement System)

The measurement system according to an embodiment of the present technology may further include an analysis unit that analyzes the biometric information. This will be described with reference to FIG. 13. FIG. 13 is a block diagram illustrating a configuration example of a measurement system 1000g according to an embodiment of the present technology.

As illustrated in FIG. 13, the measurement system 1000g includes an analysis unit 7 that analyzes the biometric information generated by the biometric information generation unit 6. The analysis unit 7 is implementable by, for example, a computing unit such as a CPU reading a program. The analysis unit 7 is configured to, for example, perform an analysis of a heart rate, or variation in the heart rate, or the like, and evaluate a stress or non-stress state, sleeping quality, or the like. In addition, the analysis unit 7 is configured to give a diagnosis of a heart disease, an autonomic nerve disease, or the like by using the heart rate, the variation in the heart rate, or the like. A publicly known technique may be used to perform such an analysis.

The biometric information generation unit 6 may be included in the cloud server 200, or may be included in the measurement device 100.

Alternatively, the biometric information generation unit 6 may be included in a terminal 400 coupled to the cloud server 200 via the information communication network 300. The terminal 400 is configured to display a result of the analysis performed by the analysis unit 7. The terminal 400 may be, for example, a personal computer, a smartphone terminal, a tablet terminal, a mobile phone terminal, a PDA (Personal Digital Assistant), a wearable terminal (HMD: Head Mounted Display such as an eyeglass-type HMD, a watch-type terminal, or a band-type terminal), or the like.

The contents described above regarding the measurement system according to the eighth embodiment of the present technology are applicable to other embodiments of the present technology unless there is any technical inconsistency.

### 9. Ninth Embodiment of Present Technology (Example 9 of Measurement System)

When the body motion information and the authentication information are associated with each other, it is preferable that the body motion information and the authentication information are prevented from being erroneously associated. For example, it is preferable to prevent association of body motion information related to a subject A and authentication information related to a subject B.

To achieve this, it is preferable to display the image acquired by the acquisition unit 3 through imaging, and to display the authentication information (such as the name or the like of the subject) at a position at which the subject is captured.

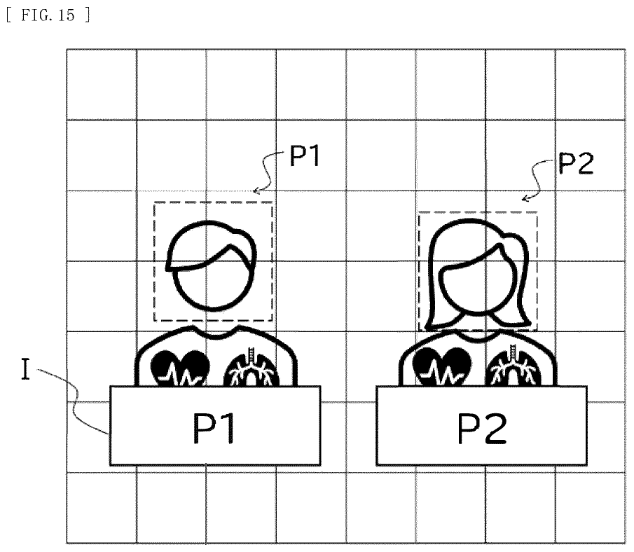

FIG. 14 is a block diagram illustrating a configuration example of a measurement system 1000h according to an embodiment of the present technology. As illustrated in FIG. 14, the measurement system 1000h may further include a display unit 8 that displays the image acquired by the acquisition unit through imaging. The display unit 8 may be, for example, a display device, a projector device, or the like.

An example of the image to be displayed by the display unit 8 will be described with reference to FIG. 15. FIG. 15 is an example of the image to be displayed by the display unit 8 according to an embodiment of the present technology. As illustrated in FIG. 15, the display unit 8 displays authentication information I at each of positions at which the subjects P1 and P2 are displayed. This allows the subject to check whether the association is made appropriately, and to make a correction in a case where the association is made erroneously.

It is to be noted that as described above, the acquisition unit 3 preferably refrains from acquiring the second position information of the subject for whom the personal authentication has not been performed, in order to prevent erroneous association. This prevents association of the body motion information and the authentication information for the subject for whom the personal authentication has not been performed. As a result, it is possible to prevent erroneous association.

It is to be noted that an instruction regarding an action may be given to the subject via the display unit 8. For example, a text to instruct the subject to do a particular exercise may be displayed on the display unit 8. The generation unit 2 is configured to generate the body motion information of the subject who is doing the exercise.

The contents described above regarding the measurement system according to the ninth embodiment of the present technology are applicable to other embodiments of the present technology unless there is any technical inconsistency.

### 10. Tenth Embodiment of Present Embodiment (Example 10 of Measurement System)

The acquisition unit 3 preferably follows a movement of the subject. This allows the acquisition unit 3 to acquire the second position information regarding the position of the subject and the authentication information based on the personal authentication of the subject even if the position of each of a plurality of subjects changes.

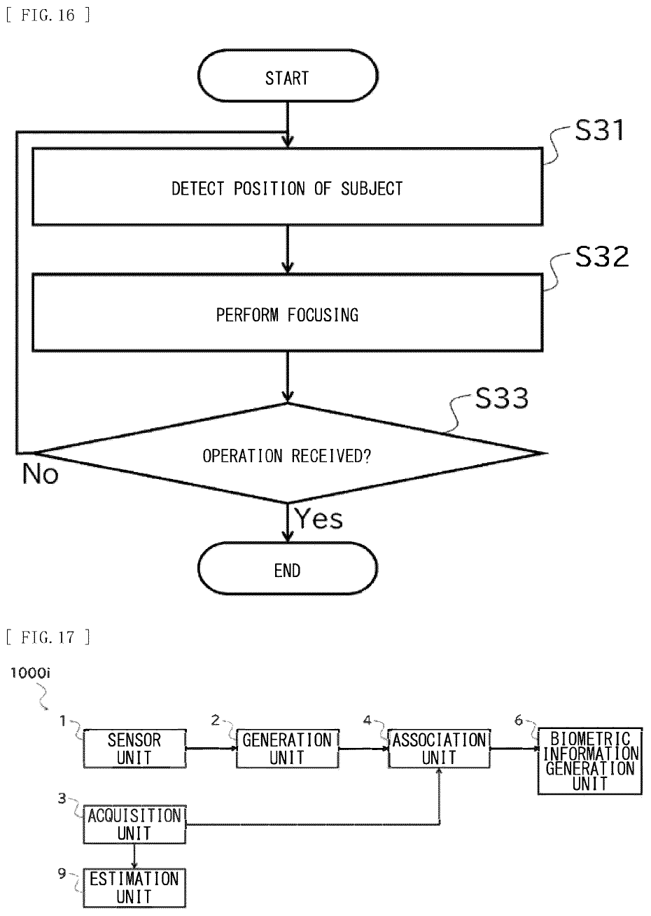

This will be described with reference to FIG. 16. FIG. 16 is a flowchart illustrating an example of the procedure of the measurement system 1000 according to the embodiment of the present technology.

First, in step S31, the acquisition unit 3 detects the position of the subject. A method of performing this detection is not particularly limited. For example, a technique such as face recognition of the subject may be used.

Thereafter, in step S32, the acquisition unit 3 performs focusing. For example, the acquisition unit 3 measures the distance to the subject and performs focusing by an autofocusing function.

The processes in step S31 and step S32 are repeated until a predetermined operation is received. This allows the acquisition unit 3 to follow the movement of the subject.

For example, when a predetermined operation such as turning off of a power source is received (step S33: Yes), the acquisition unit 3 ends the following of the movement of the subject.

As with the acquisition unit 3, the sensor unit 1 may also follow the movement of the subject. For example, the sensor unit 1 is configured to follow the movement of the subject on the basis of a change in intensity of the received second signal.

The contents described above regarding the measurement system according to the tenth embodiment of the present technology are applicable to other embodiments of the present technology unless there is any technical inconsistency.

### 11. Eleventh Embodiment of Present Technology (Example 11 of Measurement System)

The measurement system 1000 according to the embodiment of the present technology may further include an estimation unit that estimates the biometric information on the basis of the image data acquired by the acquisition unit 3 through imaging. This will be described with reference to FIG. 17. FIG. 17 is a block diagram illustrating a configuration example of a measurement system 1000i according to an embodiment of the present technology. As illustrated in FIG. 17, the measurement system 1000 includes an estimation unit 9 that estimates the biometric information on the basis of the image data acquired by the acquisition unit 3 through imaging. The estimation unit 9 is implementable by, for example, a computing unit such as a CPU reading a program. It is possible to analyze the biometric information with higher accuracy by analyzing this biometric information and the biometric information generated by the biometric information generation unit 6 in combination.

A method of estimating the biometric information on the basis of the image data is not particularly limited. For example, a face recognition technique, a blood flow measurement technique, a heart rate measurement technique, an attitude estimation technique, or the like may be used.

The face recognition technique is a technique of estimating the gender, the age, an emotional state, a fatigue level, or the like of a person on the basis of the shape of the face, the outline of the face, the position of the eye, the size of the eye, the shape of the mouth, or the like.

The blood flow measurement technique is a technique of estimating a blood flow by using an infrared ray camera, a thermal imaging camera, or the like to measure a change in temperature of a part where the blood flows.

The heart rate measurement technique is a technique of measuring a heart rate by detecting a slight change in color of a surface of the face, the finger, or the like.

The attitude estimation technique is a technique of estimating a fatigue level, a stress level, an amount of exercise, or the like on the basis of the attitude of the subject.

The contents described above regarding the measurement system according to the eleventh embodiment of the present technology are applicable to other embodiments of the present technology unless there is any technical inconsistency.

### 12. Twelfth Embodiment of Present Technology (Example of Measurement Device)

The present technology provides a measurement device that includes the sensor unit, the generation unit, and the acquisition unit. The sensor unit transmits the first signal to the subject, and receives the second signal reflected by the subject. The generation unit generates the body motion information regarding the body motion of the subject and the first position information regarding the position of the subject on the basis of the first signal and the second signal. The acquisition unit acquires the second position information regarding the position of the subject and the authentication information based on the personal authentication of the subject.

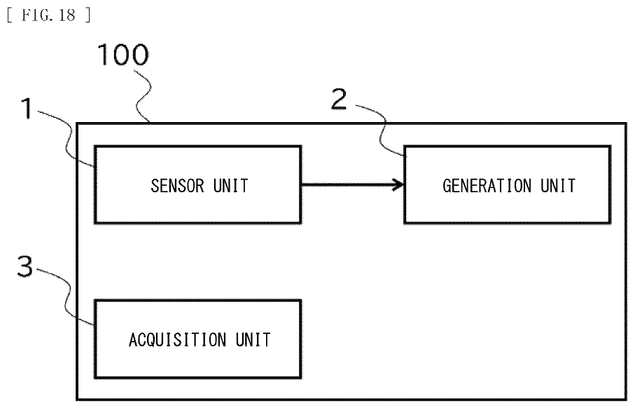

A configuration example of the measurement device will be described with reference to FIG. 18. FIG. 18 is a block diagram illustrating a configuration example of a measurement device 100 according to an embodiment of the present technology. As illustrated in FIG. 18, the measurement device 100 includes the sensor unit 1, the generation unit 2, and the acquisition unit 3.

The sensor unit 1 transmits the first signal to the subject, and receives the second signal reflected by the subject. It suffices that the first signal and the second signal allow for acquisition of at least the distance to the subject or the direction in which the subject is present. The first signal and the second signal are thus not particularly limited in kind of signal.

The generation unit 2 generates the body motion information regarding the body motion of the subject and the first position information regarding the position of the subject on the basis of the first signal and the second signal. The body motion information includes the information regarding the minute motion of the body tissue of the subject, the information regarding the minute motion of the surface of the body of the subject, and the like. The first position information includes at least the information regarding the distance to the subject and the information regarding the direction of the subject.

The acquisition unit 3 acquires the authentication information based on the personal authentication of the subject, and the second position information regarding the position of the subject. The second information acquired by a method different from that of the sensor unit 1 includes at least the information regarding the direction of the subject.

It is possible to associate the body motion information and the authentication information with each other by collating the first position information and the second position information with each other. Such association may be performed by the measurement device 100, or may be performed in a cloud server or the like outside the measurement device 100.

Such association makes it possible to simultaneously measure the biometric information while simultaneously performing the personal authentication for a plurality of subjects. This improves user-friendliness and improves measurement efficiency.

The contents described above regarding the measurement system according to the twelfth embodiment of the present technology are applicable to other embodiments of the present technology unless there is any technical inconsistency.

### 13. Thirteenth Embodiment of Present Technology (Example of Measurement Method)

The present technology provides a measurement method including: transmitting the first signal to the subject and receiving the second signal reflected by the subject; generating the body motion information regarding the body motion of the subject and the first position information regarding the position of the subject on the basis of the first signal and the second signal; acquiring the second position information regarding the position of the subject and the authentication information based on the personal authentication of the subject; and associating the body motion information and the authentication information with each other by collating the first position information and the second position information with each other.

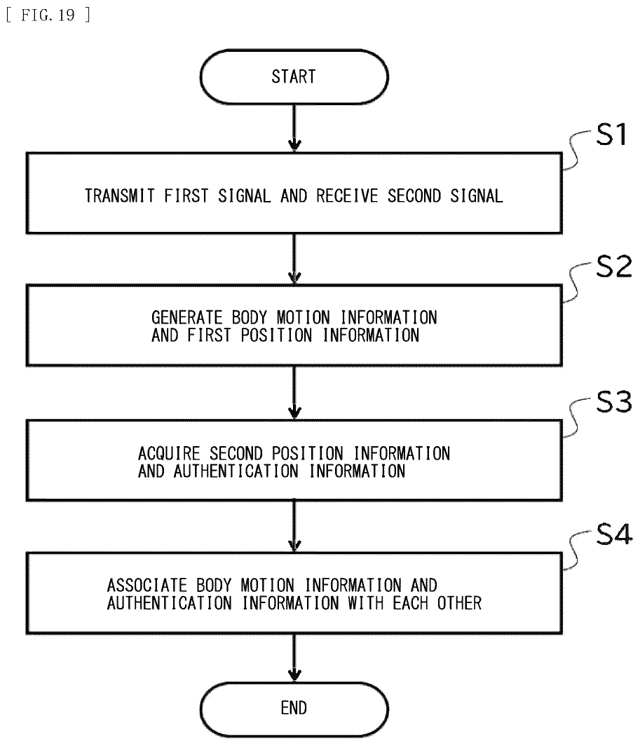

An example of a procedure of the measurement method will be described with reference to FIG. 19. FIG. 19 is a flowchart illustrating an example of the procedure of the measurement method according to an embodiment of the present technology.

As illustrated in FIG. 19, first, in step S1, the first signal is transmitted to the subject and the second signal reflected by the subject is received. For example, this is achievable by using the sensor unit 1 according to any of the other embodiments described above.

Thereafter, in step S2, the body motion information regarding the body motion of the subject and the first position information regarding the position of the subject are generated on the basis of the first signal and the second signal. For example, this is achievable by using the generation unit 2 according to any of the other embodiments described above.

Thereafter, in step S3, the second position information regarding the position of the subject and the authentication information based on the personal authentication of the subject are acquired. For example, this is achievable by using the acquisition unit 3 according to any of the other embodiments described above.

Thereafter, in step S4, the body motion information and the authentication information are associated with each other by collating the first position information and the second position information with each other. For example, this is achievable by using the association unit 4 according to any of the other embodiments described above.

The contents described above regarding the measurement system according to the thirteenth embodiment of the present technology are applicable to other embodiments of the present technology unless there is any technical inconsistency.

It is to be noted that an embodiment according to the present technology is not limited to each of the embodiments described above, and various modifications may be made without departing from the gist of the present technology. The specific numerical values, shapes, materials (including compositions), and the like described in each of the embodiments are merely examples, and are non-limiting.

In addition, the present technology may have any of the following configurations.

[1]

A measurement system including:
a sensor unit that transmits a first signal to a subject and receives a second signal reflected by the subject;
a generation unit that generates body motion information regarding body motion of the subject and first position information regarding a position of the subject on the basis of the first signal and the second signal;
an acquisition unit that acquires authentication information based on personal authentication of the subject, and second position information regarding the position of the subject; and
an association unit that associates the body motion information and the authentication information with each other by collating the first position information and the second position information with each other.
   [2]

The measurement system according to [1], in which the first position information includes at least information regarding a distance to the subject and information regarding a direction of the subject.

[3]

The measurement system according to [1] or [2], in which each of the first signal and the second signal is a signal that is of a millimeter-wave band and has a frequency varying over time.

[4]

The measurement system according to any one of [1] to [3], in which the sensor unit includes a plurality of reception antennas, the reception antennas each receiving the second signal.

[5]

The measurement system according to any one of [1] to [4], in which the second position information includes at least information regarding a direction of the subject, and
the acquisition unit acquires the information regarding the direction of the subject on the basis of an image obtained through imaging of the subject.
   [6]

The measurement system according to any one of [1] to [5], in which the acquisition unit refrains from acquiring the second position information of the subject for whom the personal authentication has not been performed.

[7]

The measurement system according to any one of [1] to [6], in which
the second position information includes at least information regarding a distance to the subject and information regarding a direction of the subject, and
the acquisition unit performs imaging of the subject to obtain a plurality of stereo camera images having parallax, and acquires the information regarding the distance to the subject and the information regarding the direction of the subject on the basis of the stereo camera images.
   [8]

The measurement system according to any one of [1] to [7], in which
the second position information includes at least information regarding a distance to the subject and information regarding a direction of the subject, and
the acquisition unit transmits a third signal to the subject, receives a fourth signal reflected by the subject, and acquires the information regarding the distance to the subject and the information regarding the direction of the subject on the basis of the third signal and the fourth signal.
   [9]

The measurement system according to any one of [1] to [8], in which the acquisition unit includes a LiDAR.

[10]

The measurement system according to any one of [1] to [9], in which the acquisition unit includes a single camera and a ToF sensor.

[11]

The measurement system according to any one of [1] to [10], in which the acquisition unit acquires the authentication information by performing face recognition of the subject.

[12]

The measurement system according to any one of [1] to [11], in which the generation unit and the association unit are coupled to each other via an information communication network.

[13]

The measurement system according to any one of [1] to [12], further including a biometric information generation unit that generates biometric information of the subject on the basis of parameter information and the body motion information, the parameter information being adapted to generate the first signal.

[14]

The measurement system according to [13], in which the generation unit and the biometric information generation unit are coupled to each other via an information communication network.

[15]

The measurement system according to [13] or [14], in which the parameter information and the body motion information are separately transmitted to the biometric information generation unit via an information communication network.

[16]

The measurement system according to any one of [13] to [15], further including an analysis unit that analyzes the biometric information generated by the biometric information generation unit.

[17]

The measurement system according to any one of [1] to [16], further including
a display unit that displays an image acquired by the acquisition unit through imaging, in which
the display unit displays the authentication information at a position at which the subject is displayed.
   [18]

The measurement system according to any one of [1] to [17], in which the acquisition unit follows a movement of the subject.

[19]

The measurement system according to any one of [1] to [18], further including an estimation unit that estimates biometric information on the basis of image data acquired by the acquisition unit through imaging.

[20]

A measurement apparatus including:
a sensor unit that transmits a first signal to a subject and receives a second signal reflected by the subject;
a generation unit that generates body motion information regarding body motion of the subject and first position information regarding a position of the subject on the basis of the first signal and the second signal; and
an acquisition unit that acquires second position information regarding the position of the subject, and authentication information based on personal authentication of the subject.
   [21]

A measurement method including:
transmitting a first signal to a subject and receiving a second signal reflected by the subject;
generating body motion information regarding body motion of the subject and first position information regarding a position of the subject on the basis of the first signal and the second signal;
acquiring second position information regarding the position of the subject, and authentication information based on personal authentication of the subject; and
associating the body motion information and the authentication information with each other by collating the first position information and the second position information with each other.

### REFERENCE SIGNS LIST

1000 measurement system
1 sensor unit
11 transmission antenna
12 reception antenna
2 generation unit
3 acquisition unit
31 right camera
32 left camera
33 camera
34 Tof sensor
4 association unit
5 AD converter
6 biometric information generation unit
7 analysis unit
8 display unit
9 estimation unit
100 measurement device
200 cloud server
300 information communication network
400 terminal
S1 transmitting a first signal and receiving a second signal
S2 generating body motion information and first position information
S3 acquiring second position information and authentication information
S4 associating body motion information and authentication information

1. A measurement system comprising:
a sensor unit that transmits a first signal to a subject and receives a second signal reflected by the subject;
a generation unit that generates body motion information regarding body motion of the subject and first position information regarding a position of the subject on a basis of the first signal and the second signal;
an acquisition unit that acquires authentication information based on personal authentication of the subject, and second position information regarding the position of the subject; and
an association unit that associates the body motion information and the authentication information with each other by collating the first position information and the second position information with each other.

2. The measurement system according to claim 1, wherein the first position information includes at least information regarding a distance to the subject and information regarding a direction of the subject.

3. The measurement system according to claim 1, wherein each of the first signal and the second signal is a signal that is of a millimeter-wave band and has a frequency varying over time.

4. The measurement system according to claim 1, wherein the sensor unit includes a plurality of reception antennas, the reception antennas each receiving the second signal.

5. The measurement system according to claim 1, wherein
the second position information includes at least information regarding a direction of the subject, and
the acquisition unit acquires the information regarding the direction of the subject on a basis of an image obtained through imaging of the subject.

6. The measurement system according to claim 1, wherein the acquisition unit refrains from acquiring the second position information of the subject for whom the personal authentication has not been performed.

7. The measurement system according to claim 1, wherein
the second position information includes at least information regarding a distance to the subject and information regarding a direction of the subject, and
the acquisition unit performs imaging of the subject to obtain a plurality of stereo camera images having parallax, and acquires the information regarding the distance to the subject and the information regarding the direction of the subject on a basis of the stereo camera images.

8. The measurement system according to claim 1, wherein
the second position information includes at least information regarding a distance to the subject and information regarding a direction of the subject, and
the acquisition unit transmits a third signal to the subject, receives a fourth signal reflected by the subject, and acquires the information regarding the distance to the subject and the information regarding the direction of the subject on a basis of the third signal and the fourth signal.

9. The measurement system according to claim 1, wherein the acquisition unit includes a single camera and a ToF sensor.

10. The measurement system according to claim 1, wherein the acquisition unit acquires the authentication information by performing face recognition of the subject.

11. The measurement system according to claim 1, wherein the generation unit and the association unit are coupled to each other via an information communication network.

12. The measurement system according to claim 1, further comprising a biometric information generation unit that generates biometric information of the subject on a basis of parameter information and the body motion information, the parameter information being adapted to generate the first signal.

13. The measurement system according to claim 12, wherein the generation unit and the biometric information generation unit are coupled to each other via an information communication network.

14. The measurement system according to claim 12, wherein the parameter information and the body motion information are separately transmitted to the biometric information generation unit via an information communication network.

15. The measurement system according to claim 12, further comprising an analysis unit that analyzes the biometric information generated by the biometric information generation unit.

16. The measurement system according to claim 1, further comprising
a display unit that displays an image acquired by the acquisition unit through imaging, wherein
the display unit displays the authentication information at a position at which the subject is displayed.

17. The measurement system according to claim 1, wherein the acquisition unit follows a movement of the subject.

18. The measurement system according to claim 1, further comprising an estimation unit that estimates biometric information on a basis of image data acquired by the acquisition unit through imaging.

19. A measurement device comprising:
a sensor unit that transmits a first signal to a subject and receives a second signal reflected by the subject;
a generation unit that generates body motion information regarding body motion of the subject and first position information regarding a position of the subject on a basis of the first signal and the second signal; and
an acquisition unit that acquires second position information regarding the position of the subject, and authentication information based on personal authentication of the subject.

20. A measurement method comprising:
transmitting a first signal to a subject and receiving a second signal reflected by the subject;
generating body motion information regarding body motion of the subject and first position information regarding a position of the subject on a basis of the first signal and the second signal;
acquiring second position information regarding the position of the subject, and authentication information based on personal authentication of the subject; and
associating the body motion information and the authentication information with each other by collating the first position information and the second position information with each other.
